(19) United States
(12) Patent Application Publication
(10) Pub. No.: US 2021/0077091 A1
(43) Pub. Date: Mar. 18, 2021

(54) SUTURE PASSER WITH LOCKING ACTUATOR

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: John A. Slusarz, Jr., Hopedale, MA (US); Joseph Johnson, Concord, MA (US); Geoffrey I. Karasic, Milton, MA (US); Timothy Young, Natick, MA (US)

(73) Assignee: Smith & Nephew, Inc., Memphis, TN (US)

(21) Appl. No.: 17/049,367

(22) PCT Filed: May 22, 2019
(86) PCT No.: PCT/US2019/033441

§ 371 Date: Oct. 21, 2020

Related U.S. Application Data
(60) Provisional application No. 62/677,228, filed on May 29, 2018.

(51) Int. Cl. A61B 17/04; A61B 17/06; A61B 90/00

(52) U.S. Cl. CPC A61B 17/0483; A61B 17/0485; A61B 17/06066; A61B 2017/00367; A61B 90/03; A61B 2017/061; A61B 17/06004

(57) ABSTRACT

Suture passer devices include a cannulated needle, a slidable member extending through the needle and a handle attached to a proximal portion of the needle. An actuator is in operative communication with a proximal portion of the slidable member. The actuator is configured to selectively translate the slidable member distally relative to the needle whereby the slidable member is extended relative to a distal portion of the needle and proximally relative to the needle whereby the slidable member is retracted and secured in a retracted position relative to the distal portion of the needle. The actuator further includes a locking mechanism for selectively locking the slidable member while in the retracted position. A first range of motion of the actuator is configured to control extending and retracting the slidable member and a second range of motion is configured to control locking and unlocking the slidable member.

### BACKGROUND

The present disclosure concerns a surgical instrument for manipulating suture. In particular, the present disclosure relates to an instrument for passing suture through tissue.

In many surgical procedures, suture is used to close wounds and may be used to repair damage to ligaments and soft tissue. As part of the repair, suture may be routed through tissues to stitch or hold the tissue together, or for the purposes of capturing the tissue and anchoring it to a surgical implant such as a suture anchor. Known instruments for suture passing typically consist of a piercing portion or needle, which may be curved, and a means for retaining the suture within a portion of the needle to enable the suture to be manipulated and passed through tissue during the repair procedure.

In example implementations, such as described in U.S. application Ser. No. 14/193,069 and U.S. application Ser. No. 15/306,406 a slidable member, e.g., a wire member or a hook member may be included within a needle lumen for facilitating retrieving/securing a suture. In operation, the slidable member may be selectively extended distally from the tip of the needle to facilitate capturing a suture and then retracted in order to secure the captured suture (i.e., by relative motion between the needle and the slidable member). The slidable member is typically controlled via an actuator on a handle of the instrument. For example, the handle may include a lever, slide, button, knob, or other type of actuator configured to selectively translate the slidable member shaft distally relative to the needle (whereby the slidable member is extended relative to a distal portion of the cannulated needle) and proximally relative to the needle (whereby the slidable member is retracted and secured in a retracted position relative to the distal portion of the cannulated needle).

In use, it is important that the slidable remain in a retracted position any time the needle is being passed through tissue. However, in some instances the slidable member may be unintentionally extended by accidental actuation during piercing (e.g., where a user accidently pushes forward on a thumb actuator while trying to pierce through tissue) or by the user not fully closing the suture retention features after grasping a suture. This can lead to the slidable member becoming snagged within the surrounding tissue resulting in damage to the tissue as well as to the instrument. For example, the slidable member can inadvertently be bent back over the needle, bent out of plane with its extension path, or become deformed in any number of different ways leading to improper functionality. Thus, there exists a need for improved suture passer instruments and methods that prevent unintentional extension of the slidable member. These and other deeds are addressed by the present disclosure.

### SUMMARY OF THE DISCLOSURE

The present disclosure seeks to overcome at least some of the above issues by providing a locking mechanism for selectively locking the slidable member, e.g., while the slidable member is in the retracted position. Advantageously, a same toggle of the actuator may be configured to enable extending, retracting, locking and unlocking the slidable member, where a different motion of the toggle is used for extending and retracting than for locking and unlocking. Notably, the use of the same toggle may simplify operation, e.g., by allowing a user to control all aspects with a single finger and without changing a grip position. In some embodiments, the motion for locking and unlocking may be configured to lie outside a typical axis for moving the instrument to pierce the needle through tissue (e.g., outside of a longitudinal axis of the instrument as defined by the needle). This, may advantageously help prevent a user from accidentally locking or unlocking the slidable member while trying to pierce through tissue. In some embodiments, the motion for locking and unlocking may include pivoting the toggle about a rotation axis, e.g., about a longitudinal axis of the instrument. In other embodiments, locking and unlocking may include depressing or translating the toggle, e.g., about an axis perpendicular to a longitudinal axis of the instrument. In some embodiments, the toggle may be biased toward a locked position, e.g., when the slidable member is in the retracted position. For example, a cantilever, spring mechanism or other biasing mechanism may maintain the toggle the locked position until a force is applied to change the position. This biasing may further ensure against accidently extending the slidable member. In example embodiments, slidable member may only be locked while in certain positions, e.g., while in a retracted position (note that in some embodiments there may be more than one retracted position, e.g., a first retracted position where a suture is held in an eyelet but is free to translate, and a second retracted position where the suture is clamped).

In example embodiments, a suture passer device is disclosed which may include a cannulated needle, a slidable member extending through the cannulated needle and a handle attached to a proximal portion of the cannulated needle and including an actuator in operative communication with a proximal portion of the slidable member. The actuator may advantageously be configured to selectively translate the slidable member distally relative to the needle whereby the slidable member is extended relative to a distal portion of the cannulated needle and proximally relative to the needle whereby the slidable member is retracted and secured in a retracted position relative to the distal portion of the cannulated needle. The actuator may further include a locking mechanism for selectively locking the slidable member while in the retracted position. Notably, a first range of motion of the actuator may be configured to control extending and retracting the slidable member and a second range of motion different from the first range of motion may be configured to control locking and unlocking the slidable member (both ranges of motion are implemented via the same actuator). In example embodiments, the first range of motion may be a translational movement of the actuator parallel with a longitudinal axis of the needle. In further example embodiments, the second range of motion may be a rotational movement of the actuator around a longitudinal axis of the needle. In other embodiments, the second range of motion may be a translation movement of the actuator parallel to an axis transverse to the longitudinal axis of the shaft.

In example embodiments, the handle may define a track providing a first translational path of the actuator wherein translation of the actuator along the translational path controls extending and retracting the slidable member. In some embodiments, the track may be defined with respect to a top surface of the handle. In further embodiments, the first translational path of the actuator may be parallel with a longitudinal axis of the cannulated needle. In some embodiments, the actuator may be in directly motion coupled to slidable member. In other embodiments, the actuator may be in indirectly motion coupled to the slidable member.

In example embodiments, the track may include an elongated slot defined by the handle and extending parallel with a longitudinal axis of the needle. Thus, the slot may be configured for receiving a sliding portion of the actuator, whereby the actor is translatably mounted within the slot. In some embodiments, the track may define a proximally facing abutment surface for preventing translational movement of the actuator along the first translational path when the actuator is in a locked position. Notably, the abutment surface may in example embodiments, be defined by a projection extending into the slot on a first side thereof or by a narrowing of the slot on a first side thereof. In some embodiments, the abutment surface may be advantageously configured to abut against a distal surface of the actuator thereby preventing translational movement along the first translational path when the actuator is in a locked position. Thus, wherein the abutment surface is defined with respect to a first side of the slot, the locked position may be where the actuator is adjacent the first side of the slot. In use the actuator may thus be configured to be moved to an unlocked position where the actuator is no longer adjacent the first side of the slot, whereby the abutment surface no longer abuts against the distal surface of the actuator thus enabling translational movement along the first translational path.

In some embodiments, the track may define a biasing mechanism configured to bias the actuator toward a locked position. For example, the track defines a biasing mechanism, wherein the biasing mechanism is a cantilever operative to bias the actuator against the first side of the slot. Notably, the cantilever may sometimes partially defines a second and opposite side of the slot. In some embodiments, the cantilever may include a projection extending into the slot on the end of a resiliently flexible lever arm. In this way, the projection may be configured to abut against and apply a force to a side of the actuator thereby biasing the actuator against the first side of the slot.

In alternative embodiments, the actuator may include a depressible button mechanism which enables toggling between locked and unlocked positions. For example in some embodiments, the depressible button mechanism may configured to interact with a depressible lever arm, the depressible lever arm defining an abutment surface that is configured abut against a surface of the actuator and prevent translational movement of the actuator along the first translational path when the actuator is in a locked position.

### BRIEF DESCRIPTION OF THE DRAWINGS

The above and other aspects, features and advantages of the disclosure will be apparent from the following more particular description of examples, as illustrated in the accompanying drawings, in which:

FIG. 1 is an example suture passer, according to the present disclosure.

FIGS. 2A and 2B are expanded views of a curved distal region of the needle of the suture passer of FIG. 1 with a slidable member in a lumen of the needle in extended and retracted positions, respectively, according to the present disclosure.

FIGS. 3A-3C depict front top and cross-sectional views, respectively, of the suture passer of FIG. 1 with the actuator alternated between “locked” and “unlocked” positions, according to the present disclosure.

FIGS. 4A and 4B depict perspective and top views, respectively, of the handle and slide track of the suture passer of FIG. 1, shown in greater detail with the actuator having been removed for clarity, according to the present disclosure.

FIGS. 5A, 5B and 5C depict perspective, sectional and component views of an alternative embodiment of an actuator which may be used in conjunction with the systems and methods herein, according to the present disclosure.

FIGS. 6A and 6B illustrate alternative embodiments utilizing resistance based locking mechanisms, according to the present disclosure

### DETAILED DESCRIPTION

In the description that follows, like components have been given the same reference numerals, regardless of whether they are shown in different examples. To illustrate an example(s) of the present invention in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and in the claims, the singular form of “a”, “an”, and “the” include plural referents unless the context clearly dictates otherwise.

As used in the specification and in the claims, for the purposes of describing and defining the invention, the terms “about” and “substantially” are used represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue.

In the context of the embodiments presented herein, it is envisioned that relative motion between any two elements may advantageously be implemented via either or both elements being moved. Thus, for example, where translation of a slidable member relative to a needle is described, this is intended to encompass embodiments where the slidable member is translated with the needle remaining stationary, the needle is translated with the slidable member remaining stationary and where both the slidable member and needle are moved at the same time. Similarly, relative motion between a surgical instrument and an actuator is intended to encompass embodiments where either or both the actuator and the body of the surgical instrument are being moved.

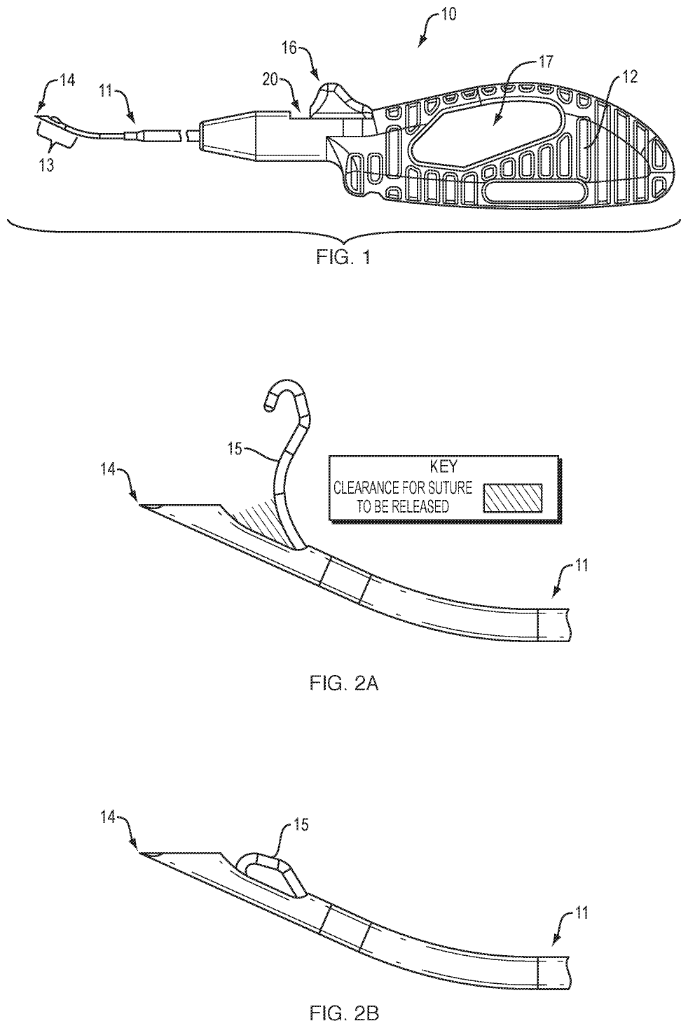

Referring to FIG. 1, there is shown an example suture passer 10 having an elongate shaft 11 extending distally from a handle 12 along a longitudinal axis of the suture passer 10. In the embodiment shown, shaft 11 includes a curved distal region 13 and pointed needle tip 14. Handle 12 is an in-line type handle. Handle 12 may, in some embodiments, include an opening 17 for accommodating a user's fingers. In alternative embodiments, the handle does not include such an opening, and the user's fingers simply fit around the handle. The handle 12 may also include an actuator 16, e.g., in the form of a thumb activated toggle.

Referring now to FIGS. 2A and 2B, the suture passer 10 includes a slidable member 15, e.g., a suture snare, which is slidably received within a lumen of shaft 11 and is extendable therefrom. According to this arrangement, and as shown in FIG. 2A, when the slidable member 15 is advanced distally by means of actuator 16, as will be described in greater detail below, a portion of the slidable member 15 projects from the tubular member, e.g., so as to enable capturing a suture (not shown). The slidable member can then be retracted, e.g., to the position shown in FIG. 2B to secure a captured suture.

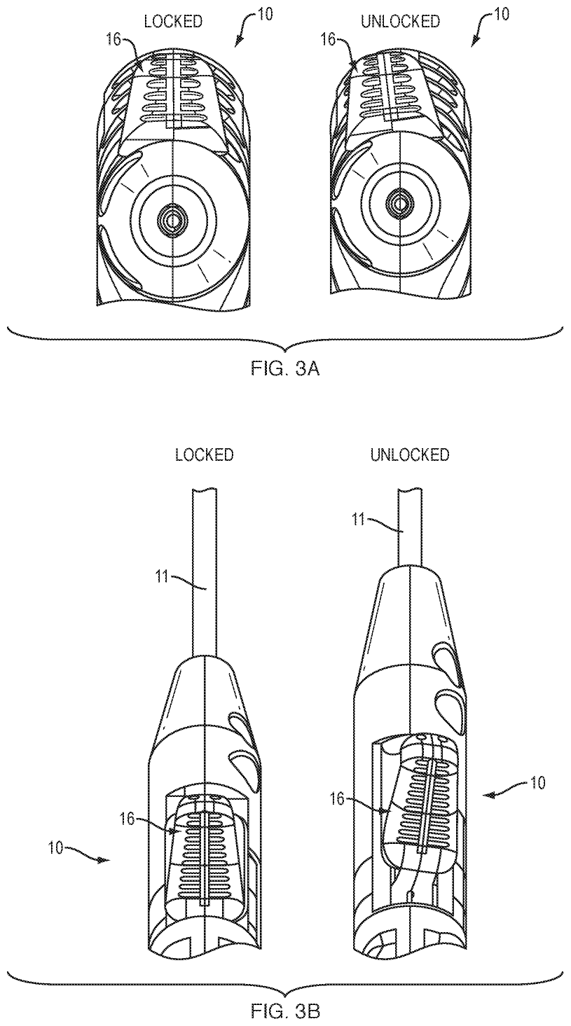

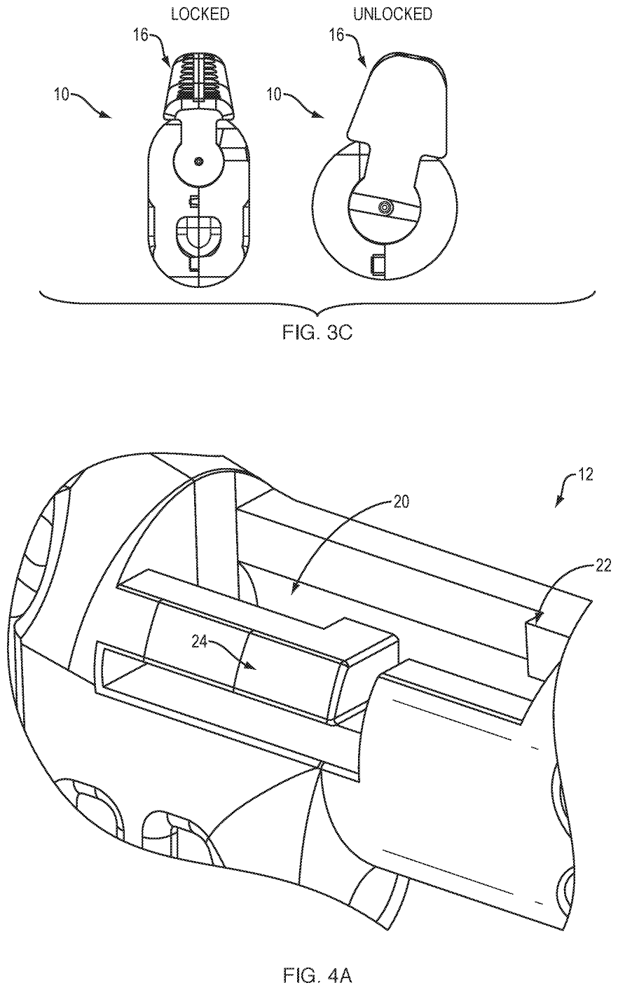

FIGS. 3A-3C depict front top and cross-sectional views, respectively, of the suture passer 10 of FIG. 1 with the actuator 16 alternated between “locked” and “unlocked” positions. It should be noted that while the depicted embodiment shows the actuator 16 centred with respect to a top surface the suture passer 10 when in the locked position and rotated (e.g., about the longitudinal axis of the shaft 11) to a side when in the unlocked position the subject application is not limited to such embodiments. Indeed, in alternative embodiments, a centred position may represent an unlocked position while a side-rotated position may represent a locked position. In yet further embodiments, the actuator 16 may be translated (e.g., along an axis transverse to the longitudinal axis of the shaft 11) instead of rotated between locked and unlocked positions. Thus, for example, the actuator may be translated from side-to-side or up/down to toggle between locked and unlocked positions.

It should be appreciated that while the locking mechanisms described herein are applied within the contact of the example suture passer 10 of FIGS. 1 and 2A-2B, the present disclosure is not limited to such implementations. Indeed, the locking mechanism described herein may be applied with respect to any actuator/toggle mechanism on a surgical instrument that effects an operation of that surgical instrument based on a first range of motion thereof. Thus, by way of example, in some embodiments, the locking mechanisms described herein may be applied with respect to an actuator that applies a first range of motion (e.g., linear or rotational motion) to a shaft/rod or other force transmission mechanism to perform operations such as opening and closing jaw or scissor elements at a distal end of the surgical instrument, extending or retracting cutting elements at a distal end of the surgical device, driving, rotating, extracting, deploying or retrieving an implant element associated with a distal end of the surgical device, etc. Thus, in some embodiments, a first range of motion of an actuator for a surgical instrument may effect an operation of the surgical instrument while a second range of motion of the same actuator may be used to toggle the actuator between locked and unlocked positions.

With reference again to FIGS. 1 and 2A-2B, in the context of the suture passer 10, the actuator 16 may be configured such that a first range of motion of the actuator 16 is operable to selectively extend and retract the slidable member 15 within shaft 11 (notably, this may be done by moving the shaft, the slidable member or both). Thus, e.g., in some embodiments, a proximal end of the slidable member 15 may be directly motion coupled to the actuator, e.g., wherein sliding/translating the actuator forward and back parallel with the longitudinal axis of the shaft is operable to translate the slidable member in the same direction/manner along the longitudinal axis of the shaft. Thus, handle 12 may define a track 20, e.g., along a top surface of the suture passer 10 for defining translational path for the actuator 16. It should be appreciated, however, that the present disclosure is not limited the depicted mechanism and translation range of motion of the actuator 16 parallel with the longitudinal axis of the shaft 11 for translating the slidable member. Indeed, it should be noted that in alternative embodiments, other mechanisms/ranges of motion of the actuator 16 may be used to effect translational movement of the slidable member. For example, in some embodiments the actuator may be configured to rotate forward and back along a transvers axis. Thus, e.g., in some embodiments, actuator 16 may be connected relative to the slidable member 15 via an indirect motion coupling, e.g., through a lever type system or gear type system. In further embodiments, the actuator could be a knob type device coupled to a rack and pinion mechanism which translates rotational movement of the knob to linear translation of the slidable member. Notably, any first range of motion of the actuator 16 may be used for effecting translation of the slidable member provided that the first range of motion of the actuator 16 is different from a second range of motion of the actuator used to toggle the actuator between locked and unlocked positions. Advantageously, however, both ranges of motion for translating the slidable member 15 and for toggling between locked and unlocked positions are implemented via the same actuator 16. In some embodiments, front-to-back rotational and/or translational movement may be used to translate the slidable member 15 while side-to-side rotational and/or translational movement may be used to effect locking/unlocking of the actuator. In some embodiments, a push down lock mechanism or a hinged lock mechanism may be implemented.

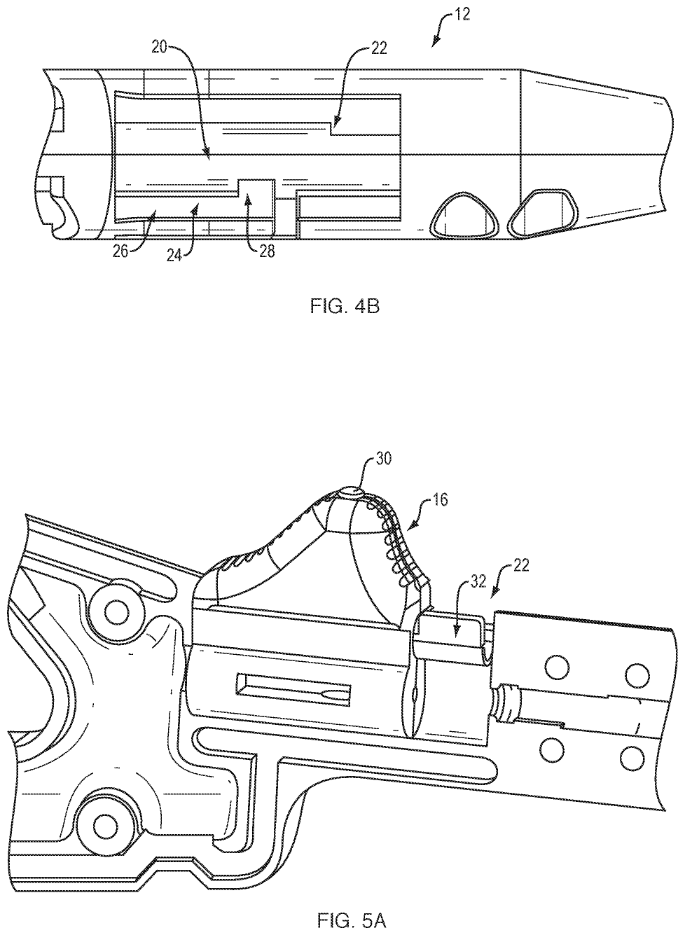

With reference now to FIGS. 4A and 4B, perspective and top views, respectively, of handle 12 and track 20 are shown in greater detail with the actuator having been removed for clarity. In the depicted embodiment, track 20 is defined as an elongated slot in the handle 12 extending parallel with the longitudinal axis of the shaft 11. The slot may be configured for receiving a sliding portion of the actuator 16, whereby the actuator 16 may be translatably mounted within the slot. The sliding portion of the actuator 16 may then be operatively coupled to a proximal end of the slidable member 15 such that translation of the actuator 16 within the slot results in a corresponding translational movement of the slidable member 15 within the shaft 11. In the depicted embodiment the track 20 further defines a proximally facing abutment surface 22 for preventing translational movement of the actuator 15 when the actuator is in a locked position, as described in greater detail herein. As depicted, the abutment surface 22 may be defined by a projection extending into the slot or by a narrowing of the slot on a first side thereof. Thus, in some embodiments, where the actuator 16 is in a locked position adjacent the first side of the slot, the abutment surface 22 may be configured to abut against a distal surface of actuator 16 thereby preventing translational movement of the actuator along track 20. In use, the actuator may selectively be moved (e.g., rotated or translated) to an unlocked position where the actuator is no longer adjacent the first side of the slot. In this position, the abutment surface 22 may no longer abut against the actuator thereby allowing the actuator 16 to translate along track 20. It is noted that the subject application is not limited to an abutment surface 22 providing means for inhibiting translational movement. Indeed, other locking mechanisms may also be utilized including, e.g., a projection and notch type configuration where a projection on the actuator interacts with a notch in a wall of the track or a notch in the actuator interacts with a projection from a wall of the track to prevent translational movement of the actuator in the slot when the actuator is in a locked position. Furthermore, in some embodiments, e.g., where an up/down motion is used to toggle between locked and unlocked positions, an upper or lower portion of the track may define an abutment surface 22, e.g., where depressing/pressing the actuator in a downward direction may result in locking/unlocking of the actuator (such as by abutting against or releasing from the abutment surface 22). In some embodiments the actuator 16 may be locked/unlocked via an electro-mechanical mechanism such as a solenoid, e.g., which may be activated via a particular range of motion of the actuator. Other locking mechanism may include detent/ratchet type features.

It should be appreciated that in some embodiments, a biasing mechanism may be used to bias the actuator into a locked or unlocked position. Thus, e.g., as depicted in FIGS. 4A and 4B, the handle 20 may in some embodiments further define cantilever 24 which be operative to bias the actuator 16 against a side wall of the track 20 with the abutment surface 22. The cantilever 24 may further partially define a second and opposite side wall of the track 20. As depicted, the cantilever 24 may include a projection 26 extending into the slot on the end of a resiliently flexible lever arm 28 which may be operatively coupled to or defined by the handle 12. In use, the projection 26 may be configured to abut and apply a force to side of the actuator 16 thereby biasing the actuator 16 against a first side of the track 20 opposite the lever arm 28. Notably, the subject application is not limited to the specific use of a cantilever mechanism as a biasing mechanism. Indeed other biasing means may also be used, e.g., springs, elastic bands, metal flexure etc. Moreover, while the depicted biasing mechanism using compressive forces to bias the actuator 16 (e.g., by pushing the actuator against a side wall) it is appreciated that in alternative embodiments, a biasing mechanism may apply tension forces instead (e.g., to pull the actuator against a side wall). In yet further embodiments, the actuator 16 may be coupled to a slidable cantilever or spring mechanism (e.g., which moves with the actuator). This may be useful for example for up/down motions where the slidable cantilever or spring mechanism may bias the actuator in a first direction (e.g., upward) in which position the actuator may against an abutment surface 22 preventing translational movement thereof along track 20.

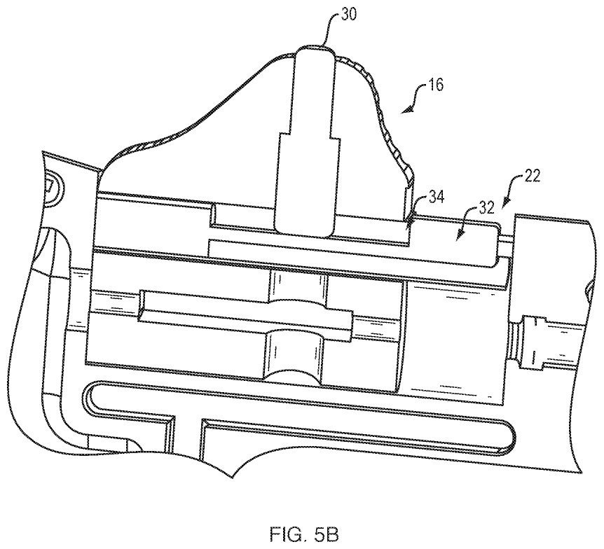

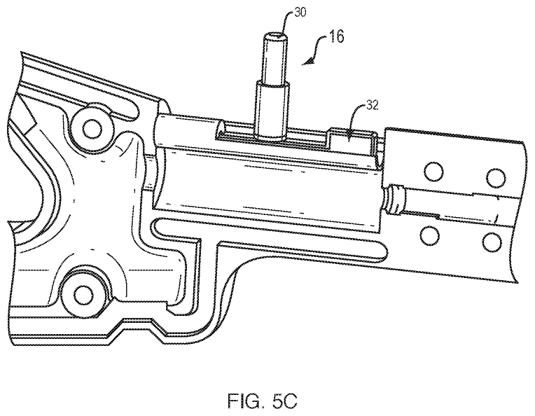

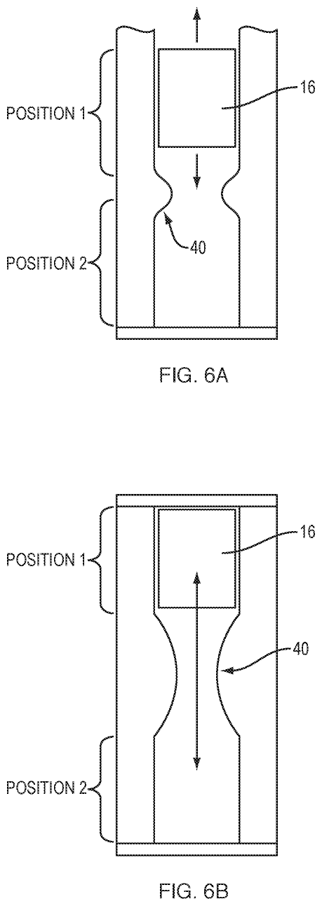

FIGS. 5A, 5B and 5C depict perspective, sectional and component views of an alternative embodiment of an actuator 16 which may be used in conjunction with the systems and methods herein. In particular, the actuator 16 in FIGS. 5A, 5B and 6C includes a depressible button mechanism 30 which enables toggling between locked and unlocked position. As depicted, the depressible button mechanism interacts 30 is configured to interact with a depressible lever arm 32. More particularly, the lever arm 32 defines an abutment surface that would abut against the actuator 16 and prevent translational movement of the actuator 16 within slot 33 unless the lever arm 32 is depressed by the button mechanism 30.

Advantageously, in some embodiments, the button mechanism may be biased, e.g., via a spring mechanism or the like in an upward direction. Notably, the subject application is not limited to the depicted embodiments of the button mechanism interacting with a lever arm. For example, in some embodiments the button mechanism may activate an electro-mechanical mechanism such as a solenoid, e.g., which may be selectively lock/unlock the actuator 16. In other embodiments, the button mechanism may instead interact to selectively couple/decouple the actuator mechanism from the slidable member 15, e.g., via a notch and projection type interaction. In yet further embodiments, the button mechanism may be used to bias the actuator 16 in a first direction, e.g., upwards so as to abut against an abutment surface. The actuator may then be depressed relative to the button mechanism (e.g., in a downward direction) to unlock translational movement.

FIGS. 6A and 6B illustrate alternative locking mechanisms that, rather than use a second range of motion to effect locking and unlocking of the actuator, use an interference or resistance based feature to resist/prevent actuation along a first range of motion until a sufficient force is applied to overcome such interference or resistance based feature. Example resistance/interference based features can include detents, ratchets, interference bumps, etc. Thus, in FIGS. 6A and 6B a resistance/interference feature 40 is included along a range of motion of an actuator 16 between first and second positions (position 1 and position 2). Advantageously, in some embodiments the resistance/interference feature 40 may allow for some degree of motion of the actuator 16 before encountering the resistance/interference feature 40 (as in the embodiment of FIG. 6A). Thus, the actuator 60 may include a partial range of motion that does not require overcoming resistance forces of the feature 40 and a partial range of motion that does require overcoming resistance forces of the feature 40. Alternatively, the resistance/interference feature 40 may resist/inhibit any degree of motion of the actuator 16 until a sufficient force is applied (as in the embodiment of FIG. 6A. In some embodiments, the resistance/interference feature 40 may be directionally specific. Thus for example, in some embodiments, the resistance/interference feature may resist motion of the actuator 16 in a first direction (e.g. from position 1 to position 2) while providing less resistance or no resistance for motion of the actuator 16 in a second direction (e.g., from position 2 to position 1). Thus, e.g., where actuator 16 is associated with a suture passer such as suture passer 10 of FIGS. 1 and 2A-2B the resistance/interference feature 40 may be configured to resist extension of the slidable member but not resist retraction of the slidable member. One possible mechanism for achieving such would be a ratchet type mechanism, detent or resistance bump feature with a steeper slope and therefor greater resistance on one side thereof.

These and other features and characteristics, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of claims.

1-24. (canceled)

25. A suture passer device comprising:
an elongated, cannulated needle having a proximal end, a distal end, and a longitudinal axis extending therebetween;
a slidable, suture-capture member extending through the needle; and
an in-line handle attached to the proximal end of the needle, the handle including an actuator in operative communication with a proximal portion of the suture-capture member and configured to slide the suture-capture member distally relative to the needle, whereby the suture-capture member is extended relative to the distal end of the needle, and proximally relative to the needle, whereby the suture-capture member is retracted and secured in a retracted position relative to the distal end of the needle;
wherein the handle defines a linear track providing a path of the actuator such that linear movement of the actuator along the path controls the extending and retracting of the suture-capture member; and
wherein the linear track includes an elongated slot defined along a top surface of the handle and extending parallel with the longitudinal axis of the needle, the slot configured for receiving a sliding portion of the actuator.

26. The suture passer device of claim 25, wherein the distal end of the needle comprises a curved distal region.

27. The suture passer device of claim 25, wherein the distal end of the needle comprises a pointed needle tip.

28. The suture passer device of claim 25, wherein the suture-capture member comprises a single, hooked member.

29. The suture passer device of claim 25, wherein the track defines a proximally facing abutment surface for preventing movement of the actuator along the path.

30. The suture passer device of claim 25, wherein the actuator is directly motion coupled to slidable member.

31. The suture passer device of claim 25, wherein the actuator is a thumb activated toggle.

32. The suture passer device of claim 25, wherein the slot includes a narrowed portion relative to a wider portion of the slot.

33. The suture passer device of claim 25, further comprising an interference or resistance based feature to resist sliding of the suture-capture member relative to the needle until a sufficient force is applied to overcome such interference or resistance based feature.

34. The suture passer device of claim 25, wherein the suture-capture member is configured to capture a suture when the suture-capture member is extended relative to the distal end of the needle, and to secure the suture within the needle when the suture-capture member is retracted relative to the distal end of the needle.

35. A method of passing suture through tissue, the method comprising:
inserting a suture passer device through tissue, the suture passer device comprising:
an elongated, cannulated needle having a proximal end, a distal end, and a longitudinal axis extending therebetween;
a slidable, suture-capture member extending through the needle; and
an in-line handle attached to the proximal end of the needle, the handle including an actuator in operative communication with a proximal portion of the suture-capture member and configured to slide the suture-capture member distally and proximally relative to the needle;
wherein the handle defines a linear track providing a path of the actuator such that linear movement of the actuator along the path controls the extending and retracting of the suture-capture member; and
wherein the linear track includes an elongated slot defined along a top surface of the handle and extending parallel with the longitudinal axis of the needle, the slot configured for receiving a sliding portion of the actuator;
using the actuator, extending the suture capture member relative to the distal end of the needle to capture a suture; and
using the actuator, retracting the suture-capture member relative to the distal end of the needle to secure the suture within the needle.

36. The method of claim 35, wherein the distal end of the needle comprises a curved distal region.

37. The method of claim 35, wherein the distal end of the needle comprises a pointed needle tip.

38. The method of claim 35, wherein the suture-capture member comprises a single, hooked member.

39. The method of claim 35, wherein the track defines a proximally facing abutment surface for preventing movement of the actuator along the path.

40. The method of claim 35, wherein the actuator is directly motion coupled to slidable member.

41. The method of claim 35, wherein the actuator is a thumb activated toggle.

42. The method of claim 35, wherein the slot includes a narrowed portion relative to a wider portion of the slot.

43. The method of claim 35, further comprising an interference or resistance based feature to resist sliding of the suture-capture member relative to the needle until a sufficient force is applied to overcome such interference or resistance based feature.

44. The method of claim 35, wherein inserting the suture passer device through the tissue comprises inserting the suture passer device through the tissue with the suture capture member in a retracted position.
